(19) United States
(12) Patent Application Publication
(10) Pub. No.: US 2012/0015428 A1
(43) Pub. Date: Jan. 19, 2012

(54) METERING ROTARY NANOPUMP, METHOD OF FABRICATING SAME, AND APPLICATIONS OF SAME

(75) Inventors: Kevin T. Seale, Nashville, TN (US); John P. Wikswo, Brentwood, TN (US); David Schaffer, Nashville, TN (US); Ronald S. Reiserer, Nashville, TN (US); Scott Darby, Nashville, TN (US)

(73) Assignee: VANDERBILT UNIVERSITY, Nashville, TN (US)

(21) Appl. No.: 13/183,287

(22) Filed: Jul. 14, 2011

Related U.S. Application Data
(60) Provisional application No. 61/365,040, filed on Jul. 16, 2010.

(51) Int. Cl. C12M 1/00; B29C 65/14; F04D 3/02

(52) U.S. Cl. 435/283.1; 415/73; 156/275.5

(57) ABSTRACT

In one aspect of the present invention, a pump includes a cam having a cam shaft, wherein the cam shaft has an axis, an exterior surface and M fins spaced-apart formed on the exterior surface along the axis; and at least one fluidic flow channel having an inlet and an outlet, disposed on a substrate, wherein the at least one fluidic flow channel helically surrounds the cam shaft such that when the cam shaft driven by the cam rotates, the M fins of the cam shaft compresses the at least one fluidic flow channels to produce a wave of compression that actuates a peristaltic flow of fluid therein so as to direct a desired amount of the fluid toward one of the inlet and the outlet.

### CROSS-REFERENCE TO RELATED PATENT APPLICATION

This application claims priority to and the benefit of, pursuant to 35 U.S.C. §119(e), U.S. provisional patent application Ser. No. 61/365,040, filed Jul. 16, 2010, entitled “Metering Rotary Nanopump, Method of Fabricating Same, and Applications of Same,” by Kevin T. Seale et al., which is incorporated herein in its entirety by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [8] represents the 8th reference cited in the reference list, namely, Seale, K. T., Faley, S., Chamberlain, J. & Wikswo, J. Macro to nano: A simple method for transporting cultured cells from milliliter scale to nanoliter scale. Exp. Biol. Med. (2010).

### STATEMENT AS TO RIGHTS UNDER FEDERALLY-SPONSORED RESEARCH

This invention was made with government support under U01AI061223-05 and 1RC2DA028981-01 awarded by the National Institutes of Health of the United States, and under HDTRAI-09-1-001 awarded by the Defense Threat Reduction Agency of the United States. The government has certain rights in the invention.

### FIELD OF THE INVENTION

The present invention generally relates to nanopumps, and more particularly to a metering rotary nanopump for microfludic systems, methods of fabricating same, and relevant applications.

### BACKGROUND OF THE INVENTION

Microfluidic (MF) devices are growing more important and useful for a wide variety of scientific and engineering fields [1], but precise control of flow in the devices can be challenging. In their ultimate form as lab-on-a-chip or Micro-Total Analysis Systems (μTAS), these devices have the ability to perform complex analyses on small numbers of cells with simple automation and reduced analysis time and reagent requirements [2]. The ability to perform rapid cell-based studies from very little starting material is already a reality, and techniques become more sophisticated almost daily as the technology advances [3-8]. Clinical tests such as HIV infection stage analysis [9] and real-time glucose sensing with feedback for insulin dosing [10] are now possible. These and other valuable lab-on-a-chip experiments are strongly dependent on the ability to precisely control the flow characteristics inside the device.

There are a number of established methods for perfusing MF devices reported in the literature [11], but many have a variety of limitations, such as prohibitive size, cost or complexity; inability to pump saline media or cells; or poorly characterized performance. Fluidic drivers can be classified as external (off-chip) or internal (on-chip). External methods include syringe pumps, gravity feed and other hydrostatic pressure methods, and transpiration-based pumps that use evaporation as the fluid-driving force [12]. Examples of on-chip methods include electroosmotic [13, 14], electrokinetic [15], piezoelectric [16] linear valve peristaltic with on-chip pneumatic [17] or off-chip mechanical actuation [18] and diode pumps [2]. Each style of pumps has advantages and disadvantages that help determine its suitability for different applications. For the applications [3, 7, 8, 19-21] among the most important considerations in choosing a pump are the ability to control and maintain instantaneous flow and flow changes and/or reversals; the minimum deliverable volume, the maximum pump head pressure, and the zero-head flow rate; the ability to recirculate and combine flow sources; and the overall size, complexity, and expense of the pump, its accessories, and drivers. Large-scale integration of microfluidic systems for biological research and diagnostics require very good flow control for driving downstream devices with a range of different resistances [22]. For cell-based studies, it is also a great benefit if cells can transit the pump without risk of damage.

The extremely small dimensions of MF devices lead to serious problems in flow monitoring and control during biological experiments that even persons experienced with traditional hydraulics applications may have difficulty managing. The overall volume of MF devices used for studies of unattached cells is typically in the 10-1000 nanoliter range with cross-sectional areas as low as 100 μm [2, 5, 6]. The small cross-sectional area has a higher perfusion pressure than systems built with more familiar large-scale tubing, and the small overall device volume may mean that small errors in flow control can rapidly lead to large errors in physical displacement within the device. This often leads to high shear rates, which cause cell damage and death even if the error is transient or pulsatile. Importantly, detection of instantaneous flow in MF devices during a biological experiment is difficult. Very high flow transients can easily escape detection by an observer watching a device through microscope eyepieces since the particulate matter and even cells are able to transit the field of view so rapidly the matter cannot be tracked or in some cases even perceived. Often the evidence of a transiently high flow having occurred in the device is the accumulation of protein and nucleic acid precipitates on the device features from large numbers of ruptured cells.

Pumping strategies and mechanisms that work well in the macro world may not be appropriate for precise control of flow and fluid mixing in MF devices, or require expensive components. The compliance of these devices and their accessories (such as plastic tubing, fittings, and syringes) may leads to long system delays and large flow transients in the MF device. Depending on the experimental setup, even a seemingly harmless bump of the perfusion apparatus can cause a damaging flow transient on-chip that appears and disappears in a moment. Microfluidic methods such as electroosmotic (EO) flow [23] or pressure-driven on-chip multi-valve peristaltic pumps [17, 22] are generally an improvement but have significant limitations. EO flow can be unstable over time, is affected by media conductivity, and only works with channels of limited cross-sectional area. Multi-valve pumps require relatively sophisticated control solenoids, electronics, and regulators, costing in the hundreds of dollars per pump. Pressure-driven flow is dependent upon the height of the column of fluid or the size of the droplet driving the flow, and is difficult to turn on or off or reverse dynamically.

Because of the difficulty of instantaneous flow measurement in MF devices during experiments, significant flow transients may occur during the course of the experiment that can affect the concentrations of important reagents and shear forces on delicate cellular structures. Good control over flow in the MF device is best achieved with a pumping system that has dimensions on the same order as the device itself so the benefit of small volumes and reaction times can be realized without large “dead-space” volumes or compliances of off-chip pumping systems. With small pumps, the systematic errors due to mechanical movements of the pump translate into smaller errors in fluid velocity and flow. It is also highly desirable in many MF experiments that the pumping system allow recirculation of the on-chip fluidic volumes for reagent. Small on-chip pumps could introduce only minimal extra volume or dead space. It would also be a great benefit if pumping and recirculation of cell solutions were possible without significant cell death. While recirculating on-chip pneumatic valves are well studied in the literature [17], they often work best for shallow channels that are not well suited for transporting or pumping eukaryotic cells, and require at least three solenoid valves, a source of high pressure gas, and a controller that together can cost several hundred dollars per pump. In many applications, the ideal pump is small, on-chip, and transports fluid volumetrically as a controlled source of flow rather than pressure.

Existing methods for supplying fluid flow to MF devices may be divided into external and internal methods. Macro-level positive displacement pumps can typically pump against very large head pressures because there is no route of counter flow for the fluid. The valves in some conventional pumps do not always close completely, leading to a possible route of backflow. The operating pressure of the some conventional pump can also be increased, but the actuator sometime is a compressible gas which is slower to respond than the stainless steel cam, and may behave nonlinearly.

Many pumping methods including all external methods do not allow practical recirculation within the device. Some conventional pumps do allow recirculation, but are limited. Existing methods for integrated pumping are limited by some or all of the following four factors: head pressure, biocompatibility, pulsatility, and power and control requirements.

It is generally believed that the use of integrated pumps may expand the use of MF devices by making them simpler to operate, smaller and more self contained. Therefore a pump is needed to fulfill the major requirements for MF device, including some or all of the following factors: precise flow control, small size, modest cost, reasonable head pressure, reversibility without hysteresis, the ability to re-circulate small volumes, low power consumption, and no fluid leakage when not powered.

Therefore, a heretofore unaddressed need exists in the art to address the aforementioned deficiencies and inadequacies.

### SUMMARY OF THE INVENTION

The present invention, in one aspect, relates to a pump. In one embodiment, the pump includes a cam having a cam shaft, wherein the cam shaft has an axis, an exterior surface and M fins spaced-apart formed on the exterior surface along the axis; and at least one fluidic flow channel having an inlet and an outlet, disposed on a substrate, wherein the at least one fluidic flow channel helically surrounds the cam shaft such that when the cam shaft driven by the cam rotates, the M fins of the cam shaft compresses the at least one fluidic flow channels to produce a wave of compression that actuates a peristaltic flow of fluid therein so as to direct a desired amount of the fluid toward one of the inlet and the outlet.

In one embodiment, the cam has a non-circular cross-section.

In one embodiment, the M fins are parallely formed on the exterior surface along the axis. In another embodiment, the M fins are helically formed on the exterior surface along the axis.

In one embodiment, the at least one fluidic flow channel comprises a plurality of channels that converge at one of the inlet and the outlet and diverge at the other of the inlet and the outlet.

The at least one fluidic flow channel is biocompatible. In one embodiment, the at least one fluidic flow channel is formed of polydimethylsiloxane (PDMS). The at least one fluidic flow channel has a cross section in a geometric shape of a circle or a polygon, where the cross section has a maximal dimension in a range of about 1-100 μm.

In one embodiment, the rotation of the cam shaft is reversible, wherein when the cam shaft rotates in a reversed direction, the desired amount of the fluid is directed toward the other of the inlet and the outlet.

When the cam shaft rotates to position one of the M fins substantially against the substrate, the fin compresses the at least one fluidic flow channel to interrupt the flow of the fluid therein, thereby producing the flow of the fluid with M interruptions per revolution, wherein the produced flow of the fluid is of a pulsatile flow.

In operation, the cam shaft rotates at a constant speed or a variable speed.

The flow rate of the fluid is controllable by at least one of the size and the number of the fluidic flow channels and the rotating speed of the cam shaft.

In one embodiment, the pump further includes means for rotating the cam.

In another aspect, the present invention relates to a pump. In one embodiment, the pump has a network of linear fluidic flow channels that diverge from an outlet and converge at an inlet, disposed on a substrate; and an external threaded rod, and rotatably placed on the network of linear fluidic flow channels, such that when the threaded rod rotates, the ridge of the threaded rod compresses one or more of the fluidic flow channels to produce a wave of compression that actuates a peristaltic flow of fluid therein so as to direct a desired amount of the fluid toward one of the inlet and the outlet.

The network of linear fluidic flow channels is biocompatible. In one embodiment, the network of linear fluidic flow channels is formed of PDMS.

Each fluidic flow channels has a cross section in a geometric shape of a circle or a polygon, where the cross section has a maximal dimension in a range of about 1-100 μm.

The flow rate of the fluid is controllable by at least one of the size and the number of the linear fluidic flow channels and the pitch and the ridge width and the rotating speed of the threaded rod.

In one embodiment, each fluidic flow channel has a corresponding offset relative to the repeating helical structure of the threaded rod, wherein the offsets of the fluidic flow channels are different from one another, thereby creating phased offsets of the flow from the network.

In one embodiment, the rotation of the threaded rod is reversible, wherein when the threaded rod rotates in a reversed direction, the desired amount of the fluid is directed toward the other of the inlet and the outlet.

In one embodiment, the threaded rod is in an Acme thread form or a trapezoidal form.

In yet another aspect, the present invention relates to a pump 1100. In one embodiment, the pump has at least one fluidic flow channel having an inlet and an outlet; and means rotatably engaged with the at least one fluidic flow channel for periodically producing a wave of compression in the at least one fluidic flow channel, wherein the wave of compression actuates a peristaltic flow of fluid in the at least one fluidic flow channel so as to direct a desired amount of the fluid toward one of the inlet and the outlet.

In one embodiment, the means comprises an external threaded rod such that when the threaded rod rotates, the ridge of the threaded rod compresses the at least one fluidic flow channel to produce the wave of compression.

In another embodiment, the means a cam having a cam shaft, wherein the cam shaft has an axis, an exterior surface and M fins spaced-apart formed on the exterior surface along the axis such that when the cam shaft driven by the cam rotates, the M fins of the cam shaft compresses the at least one helical channel to produce the wave of compression.

The at least one fluidic flow channel is biocompatible.

In a further aspect, the present invention relates to a method of fabricating a pump. In one embodiment, the method includes the steps of providing a master having a silicon wafer and a photoresist layer formed of a photoresist on the silicon wafer; exposing the photoresist layer to UV light through a patterned mask to cross-link the photoresist in selected regions in accordance with the mask to define channel regions; spin-coating the master with PDMS to form a PDMS layer that covers the defined channel regions thereon to form a rectangular, ribbon-shaped section encompassing channels; and plasma-bonding the PDMS layer having the channels to a PDMS film coated on a blank wafer to encapsulate the channels.

In one embodiment, the method further includes the step of wrapping the encapsulated channels around a cam shaft in a single helical turn to form the pump, wherein the cam shaft has M fins spaced-apart formed on its exterior surface along its axis, such that when the cam shaft driven by the cam rotates, the M fins of the cam shaft compresses one or more of the encapsulated channels to produce a wave of compression that actuates a peristaltic flow of fluid therein.

In another embodiment, the method further includes the step of engaging with the encapsulated channels with an exterior threaded rod to form the pump, such that when the threaded rod rotates, the ridge of the threaded rod compresses one or more of the encapsulated channels to produce a wave of compression that actuates a peristaltic flow of fluid therein.

In one embodiment, the method also includes the step of baking and developing the defined channel regions to form a hardened, reusable master, prior to the spin-coating step.

Additionally, the spin-coating step comprises the steps of peeling off the rectangular ribbon-shaped section of the PDMS layer; placing the peeled PDMS layer inversely on a glass slide; plasma-treating the peeled PDMS layer and the PDMS film coated on the blank wafer for a period of time; boding the peeled PDMS layer and the PDMS film coated on the blank wafer together such that the channels are encapsulated; removing the glass slide; and cutting the encapsulated channels from the blank wafer to form a rectangular ribbon-shaped piece of PDMS.

In yet a further aspect, the present invention relates to a system for manipulation of cells. In one embodiment, the system includes at least a first pump and a second pump, each pump comprising at least one fluidic flow channel having an inlet and an outlet, and means rotatably engaged with the at least one fluidic flow channel for periodically producing a wave of compression in the at least one fluidic flow channel, wherein the wave of compression actuates a peristaltic flow of fluid in the at least one fluidic flow channel so as to direct a desired amount of the fluid toward one of the inlet and the outlet.

The system also includes a downstream device comprising a plurality of cell traps arranged in a square trap chamber defining a trap region thereof; and a network of binary flow splitters, wherein each side of the trap region is connected to the network of binary flow splitters that divides or gathers a flow uniformly across the entire side, wherein the network of binary flow splitters has a first input and a first output along the x direction and a second input and a second output along the y direction perpendicular to the x direction.

As assembled, the inlet and the outlet of the first pump are respectively in fluid communications with the first input and the first output of the network of binary flow splitters, and the inlet and the outlet of the second pump are respectively in fluid communications with the second input and the second output of the network of binary flow splitters, such that in operation, the first pump and the second pump drive orthogonal flow streams in the downstream device, thereby positioning cells to a desired location in the trap region.

In one embodiment, the means comprises an external threaded rod such that when the threaded rod rotates, the ridge of the threaded rod compresses the at least one fluidic flow channel to produce the wave of compression.

In another embodiment, the means a cam having a cam shaft, wherein the cam shaft has an axis, an exterior surface and M fins spaced-apart formed on the exterior surface along the axis such that when the cam shaft driven by the cam rotates, the M fins of the cam shaft compresses the at least one helical channel to produce the wave of compression.

In one aspect, the present invention relates to a system for fluidic impedance tomography (FIT). In one embodiment, the system includes an object chamber; a common channel spaced-apart surrounding the object chamber; N pumps spaced-apart coupling between the object chamber and the common channel, each pump comprising at least one fluidic flow channel having an inlet and an outlet; and means rotatably engaged with the at least one fluidic flow channel for periodically producing a wave of compression in the at least one fluidic flow channel, wherein the wave of compression actuates a peristaltic flow of fluid in the at least one fluidic flow channel so as to direct a desired amount of the fluid toward one of the inlet and the outlet; and N pressure transducers, each pressure transducer connected between the object chamber and the surrounding common channel and associated with a respective pumps for determining the pressure difference between the periphery of the object chamber and the surrounding common channel therein. The measurement of the pressure distribution P, for each of a series of flow Q′ distributions is used to reconstruct a fluidic impedance distribution within the object chamber.

In one embodiment, a pattern of flow through the object chamber is determined by the rate and direction of the flow Qᵢ driven by each pump, where 1≦i≦N, with the constraint

$${\sum }_{i = 1}^{N} {Q}_{i} = 0$$

that the fluid is incompressible and the at least one fluidic flow channel of each pump is non-distensible.

In one embodiment, the flow patterns are determined by the gradient of the pressure distribution that satisfies Laplace's equation with the boundary conditions associated with the injection or removal from the fluid and the distribution of fluidic impedance associated with the objects contained in the central chamber.

In one embodiment, the surrounding common channel has a channel depth adapted such that a fluidic impedance of the surrounding common channel is negligible relative to that of the object chamber. The N pumps are high-impedance flow sources. The N pressure transducers are stiff.

In one embodiment, the means comprises an external threaded rod such that when the threaded rod rotates, the ridge of the threaded rod compresses the at least one fluidic flow channel to produce the wave of compression.

In one another embodiment, the means a cam having a cam shaft, wherein the cam shaft has an axis, an exterior surface and M fins spaced-apart formed on the exterior surface along the axis such that when the cam shaft driven by the cam rotates, the M fins of the cam shaft compresses the at least one helical channel to produce the wave of compression.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows schematically a peristaltic metering pump according to one embodiment of the present invention.

FIG. 2 shows schematically fabrication processes of a peristaltic metering pump according to one embodiment of the present invention, (A) fabrication processes of pump channels, and (B) wrapping processes of the channels around the rod.

FIG. 3 shows schematically an experimental setup using a peristaltic metering pump according to one embodiment of the present invention.

FIG. 4 shows the pump consistency over time. A linear fit to the data shows that the stroke volume for this pump is about 3.2 nL, which is in good agreement with a predicted value of 5.4 nL if a compression fraction of 40% is assumed. Data points are the averages of four measurements at each motor RPM made every 15 minutes. Error bars are the standard deviation.

FIG. 5 shows the flow rate versus motor speed for three pumps according to embodiments of the present invention.

FIG. 6 shows 2-D kymograph plots of a pair of beads in a trap device being driven by a hand-operated rotary nanopump in a perspective view (A), a top view (B), a front view (C) and a side view (D), according to one embodiment of the present invention. Time increases with the height in the perspective plot (A). With sequential reversals of direction caused by 8 turns of the cam in alternating directions, the bead completes three complete short round trips traveling in the vicinity of the trap before coming to rest. Trap baskets are approximately 18×18 μm².

FIG. 7 shows a flow rate as a function of externally applied head pressure at a fixed motor RPM, according to one embodiment of the present invention. Dashed line indicates the Hagen-Poiseuille relation for 25 cm of 50 μm ID cylindrical tubing—a typical connection length of PEEK tubing in our lab. Dotted line indicates the perfusion pressure of a complex, multi-cellular nanobioreactor with cells (data courtesy of Dmitry Markov).

FIG. 8 shows schematically a cross-flow device driven by two pumps whose average flow directions are orthogonal to each other in an empty trap field, according to one embodiment of the present invention, (A) schematic layout of the cross-flow device showing the square trap chamber, and (B)-(E), a kymograph plot showing 2-D flow control in a perspective view (B), a top view (C), a front view (D) and a side view (E). Cells in adjacent traps are repositioned in about 12 seconds to adjacent traps two columns to the right using hand-driven pumps and a setup similar to the top panel.

FIG. 9 shows schematically a system for fluidic impedance tomography, according to one embodiment of the present invention. The sum of the pump flows between the object chamber and the outer common channel is zero.

FIG. 10 shows cells before transiting a helical pump (A) and the same group of cells after transiting the pump (B), according to one embodiment of the present invention.

FIG. 11 shows schematically a pump, according to another embodiment of the present invention, (A) a front view, a bottom view (B), a side view (C) and a perspective view (D).

### DETAILED DESCRIPTION OF THE INVENTION

The present invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom”, “upper” or “top,” and “front” or “back” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

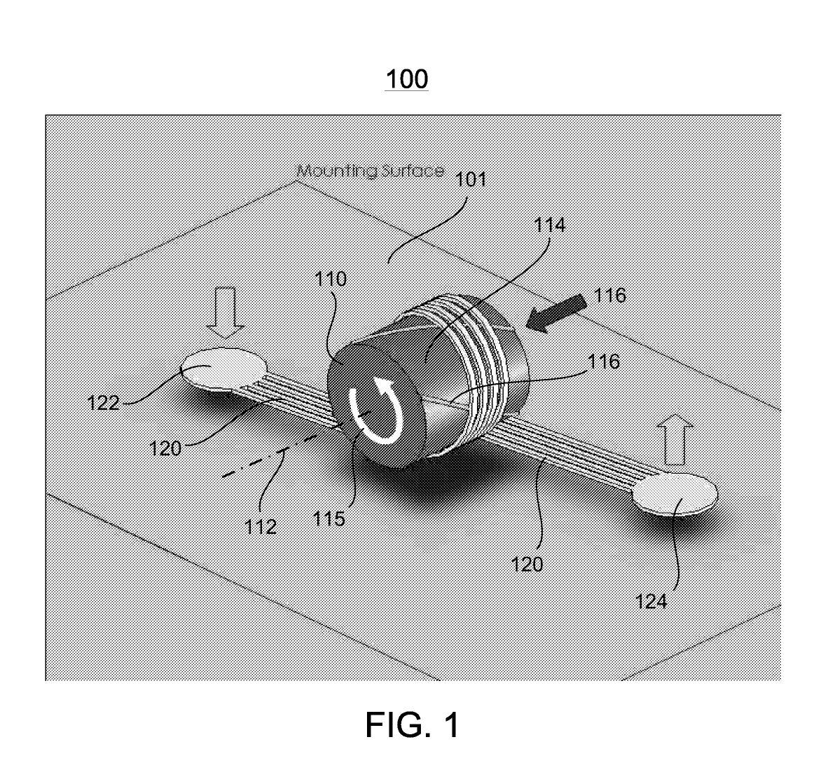

The description will be made as to the embodiments of the present invention in conjunction with the accompanying drawings in FIGS. 1-11. In accordance with the purposes of this invention, as embodied and broadly described herein, this invention, in one aspect, relates to a rotary peristaltic pump, method of manufacturing the same and applications of the same.

Referring to FIG. 1, the rotary peristaltic pump 100 is shown according to one embodiment of the present invention. The pump 100 includes a cam with a non-circular cross-section (not shown) having a cam shaft 110. The cam shaft 110 has an axis 112, an exterior surface 114 and a number of fins 116 spaced-apart formed on the exterior surface 114 along the axis 112. Preferably, the fins 116 are parallely and helically formed on the exterior surface 114 along the axis 112.

The pump 100 also includes four fluidic flow channels 120 defining a network of channels having an inlet 122 and an outlet 124. Other numbers of the fluidic flow channels 120 can also be utilized to practice the invention. The fluidic flow channels 120 are disposed on a substrate (mounting surface) 101. The channels 120 are biocompatible, and preferably, formed of PDMS. The channels 120 have a cross section in a geometric shape of a circle or a polygon, where the cross section has a maximal dimension in a range of about 1-100 μm.

As shown in FIG. 1, the fluidic flow channels 120 are helically surrounds the cam shaft 110 in one turn. As such, when the cam shaft 110 driven by the cam rotates to position one of the fins 116 substantially against the substrate 101, the fin compresses the fluidic flow channels 120 to produce a wave of compression in the channels 120 to actuate the flow of fluid therein. This will drive or direct a desired amount of the fluid toward one of the inlet 122 and the outlet 124. The amount of the fluid or the flow rate can be precisely determined by the size and number of the fluidic flow channels 120 and the rotating speed of the cam shaft 110.

The cam shaft 110 can rotate at a constant speed or a variable speed. Additionally, the rotation of the cam shaft 110 is reversible. When the cam shaft 110 rotates in one direction, the desired amount of the fluid is directed toward one of the inlet 122 and the outlet 124. When the cam shaft 110 rotates in a reversed direction, the desired amount of the fluid is directed toward the other of the inlet 122 and the outlet 124.

The rotation of the cam and thus the cam shaft 110 is driven by, but not limited to, hand-cranks, spring motors and other non-powered options in addition to simple DC or stepper motors. All of these driving methods have achieved excellent flow control, for example, by twisting the cam with human fingers. A stepper motor, capable of micro stepping, can be integrated with the pump. The spring motors which do not require electricity can be used as well.

According to the present invention, the produced flow of the fluid is of a pulsatile flow. The pulsatility of the fluid flow is determined by the number of the fins or lobes formed on the cam shaft and the number of the fluidic flow channels. For example, a single helical channel and constantly rotating cam pump produces constant flow in the channel with an interruption each time the cam shaft passes the discharge channel. A cam with M lobes/fins will produce flow with M interruptions per revolution. The track of the lobes can be helical ones. A single lobed cam may have one interruption per revolution and a bi-lobed cam may have two interruptions per revolution, etc. The flow of N lobes may thus be pulsatile, although it may be constant between interruptions. According to the present invention, the nanopump produces normally constant flow which is interrupted periodically by the rotation of the cam lobe. This “normally on” characteristic makes it possible to add the flow from multiple channels, out of phase with each other. Multiple helical channels driven by an opposite-sense helical cam should lead to phased flow interruptions in the individual channels. Recombination of the flow from the individual channels may reduce overall pulsatility in the flow.

In one embodiment, the helical metering pump can be made very small with stepper motors approximately the size of an aspirin caplet (a 12 mm cylinder, 6 mm diameter, approximately), with reduction gear heads. This makes possible the integration of multiple pumps in a single MF/BioMEMS device. One stepper motor, spring motor or hand crank may operate several pumps simultaneously as well.

The present invention offers, among other things, the possibility of loading and recirculating very small amounts of fluid, on the order of 10 microliters. Recirculation may be useful for studies of fresh blood cells in fresh plasma and other cells that are sensitive to the microenvironment. Recirculation may also increase the sensitivity of on chip analyte-specific reagent (Analyte-specific reagents (ASRs) are a class of biological molecules which can be used to identify and measure the amount of an individual chemical substance in biological specimens) reactions such as DNA hybridizations, antibody-antigen interactions and others.

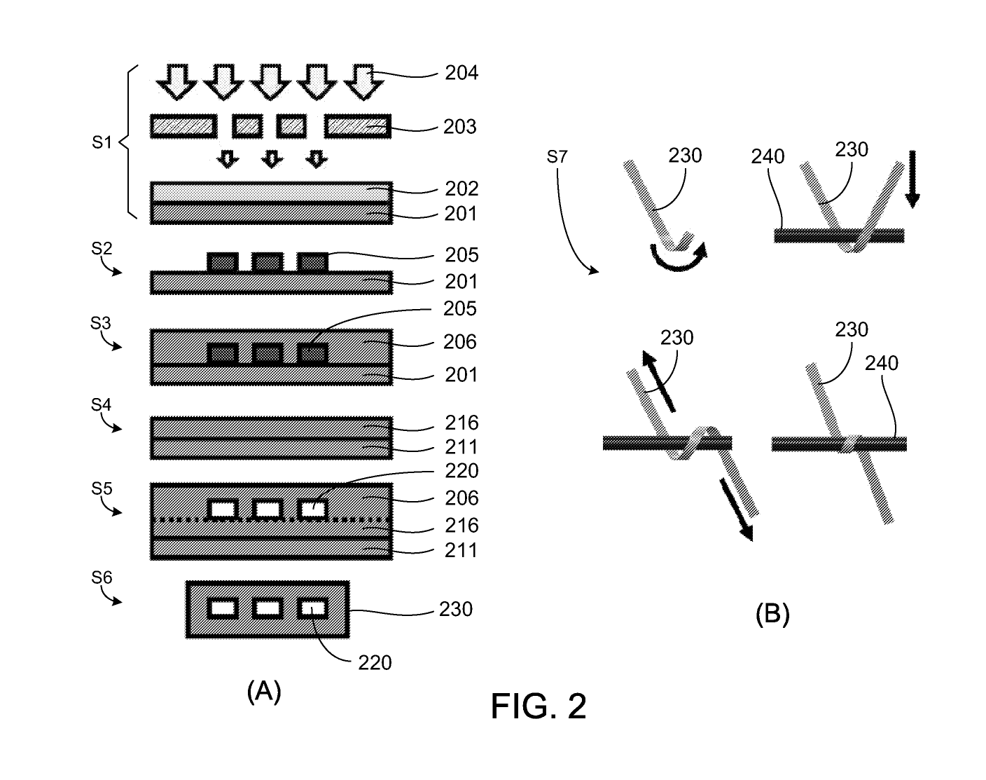

FIG. 2 shows schematically fabricating process of a pump according to one embodiment of the present invention. In one embodiment, the encapsulated channels are made from PDMS (10:1 resin: hardener) bonded to a glass slide for easy use on a microscope platform. Cylindrical stainless steel tubing is used to fabricate the helical portion of the pump, and compressed tubing with a non-circular cross-section is used as the cam. At first (step S1), a master is provided to have a silicon wafer 201 and a photoresist layer 202 formed of a photoresist on the silicon wafer 201, and the photoresist layer 202 is then exposed to UV light 204 through a patterned mask 203 to cross-link the photoresist 205 in selected regions in accordance with the mask 203 to define channel regions 205. The defined channel regions 205 are baked and developed to form a hardened, reusable master at step S2.

At step S3, the hardened, reusable master is spin-coated with PDMS to form a PDMS layer 206 that covers the defined channel regions 205 thereon, which form a rectangular, ribbon-shaped section encompassing channels 220. Then, the rectangular ribbon-shaped section of the PDMS layer 206 (205) is peeled up. The peeled PDMS layer 206 is placed inversely on a glass slide. The peeled PDMS layer 206 and a PDMS film coated on the blank wafer 211 that is formed at step S4 are plasma-treated for a period of time. At step S5, the peeled PDMS layer 206 and the PDMS film 216 coated on the blank wafer 211 are bonded together so that the channels 220 are encapsulated. Then, the glass slide is removed. A rectangular ribbon-shaped piece 230 of PDMS with the encapsulated channels 220 is obtained by cutting the bonded structure from the blank wafer 211.

The encapsulated channel piece 230 is wrapped around a cam shaft 240 in a single helical turn to form the pump at step S7.

In another embodiment, the pump was constructed using a metal compression spring concentric to a metal shaft as a casting for PDMS. The channel formed by the metal compression spring was the helical fluid channel and the through-hole left where the metal shaft had been served as the cam shaft. The cam was constructed by smashing a piece of brass tubing into an oblong cross-section. Pumping was verified by direct visual inspection of a small air bubble in the helical channel. The cross-sectional area of the helical channel is large enough to produce large flow rates, care must take to reduce the flow rates to be compatible with cellular microfluidic studies. The design and fabrication technique are useful for relatively high-flow pumps.

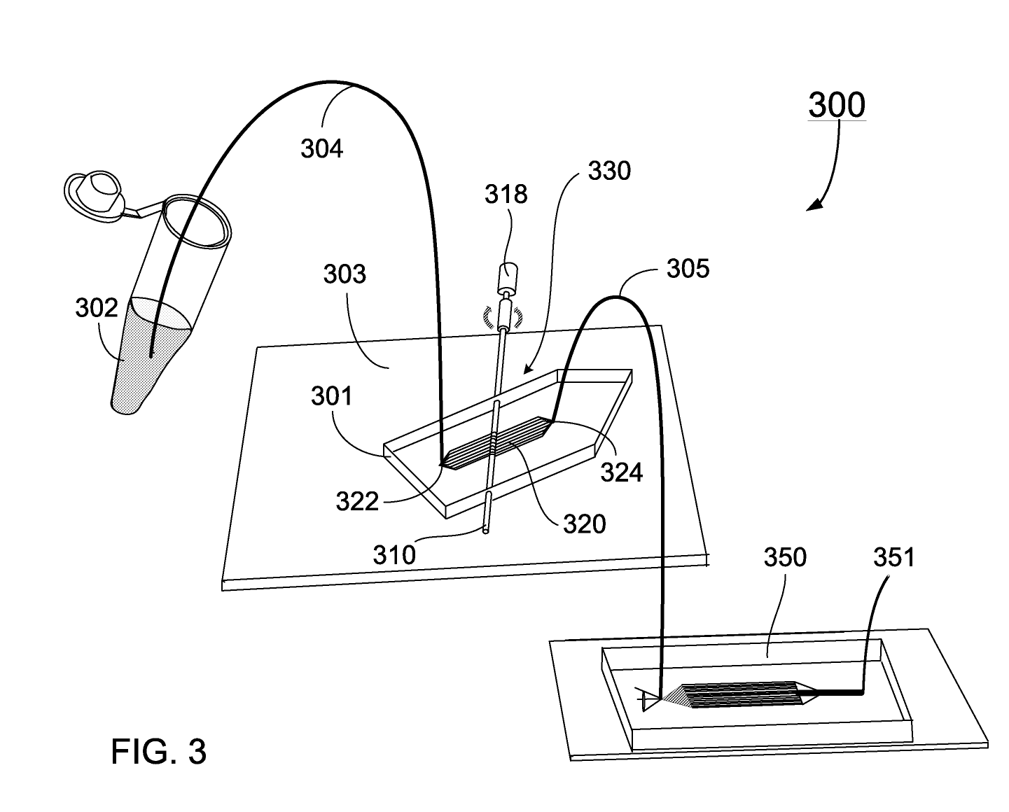

According to the present invention, such a rotary peristaltic pump fabricated with PDMS that is designed to fulfill the major requirements for MF device perfusion, including precise flow control, small size, modest cost, a reasonable head pressure, reversibility without hysteresis, the ability to recirculate small volumes, low power consumption, and no fluid leakage when not powered. FIG. 3 illustrates schematically the basic features of a pump 330 according to one embodiment of the present invention and its incorporation into a typical experimental setup. A small bore Polyetheretherketone (PEEK) tubing 304 is used to supply cells and reagents from a 1.6 mL microcentrifuge tube 302 to the pump inlet 322, while a PEEK tubing 305 is used to interconnect the outlet 324 of the pump 330 and one or more downstream devices 350. The cam 318 and thus the cam shaft 310 are rotated by hand (not shown) or spring motor, or electric motor to supply cells and reagents through the channels 320 and the PEEK tubing 304 and 305 to one or more MF devices 350. The pump 350 is mounted on a substrate 301 that is placed on a platform 303. Rotation of the cam 318 by hand or with the electric motor causes flow from the supply 302 through the pump 330 to the downstream device 350 which is reversible, and with volume delivered proportional to the number of revolutions of the rotation and the flow rate proportional to the cam revolution(s) per minute (RPM).

In one embodiment, reconnection of the outlet 351 of the MF device 350 to the inlet 322 of the pump 330 allows recirculation with very small dead space volume. The pump 330 achieves reasonable head pressure with very small stroke volumes and has a large range of output flow rates. Integration of the pump 330 with other pumps, MF devices and computer-controlled, battery-powered motors makes it an exciting addition to a growing stable of lab-on-a-chip methods and devices for exploring new frontiers in low-cost, automated systems for research and diagnostics.

According to the present invention, the fabrication of the pump is very simple. Exemplary processes of the pump fabrication are shown in FIG. 2. The fabrication in one embodiment begins by creating long parallel channels of controlled widths and heights with UV photolithography followed by soft lithography. Briefly, a master is created by spin coating a 3-inch diameter silicon wafer 201 with SU-8 negative photoresist 202 and subsequently exposing the photoresist to UV light 204 through a patterned chrome mask 203 to cross-link the negative photoresist in the channel regions 205 (at step S1). The cross-linked channels 205 are baked and developed to form a hardened, reusable master (at step S2). As an example without limitation, a channel set can have 10 parallel channels, each about 25 μm wide and about 11 μm high.

The reusable master is replica-molded with PDMS by spin-coating a very thin first layer 206 at high RPM directly onto the master (at step S3). The thickness of this layer 206 is about 20-50 μm. Precisely control this fabrication step, it is possible to achieve consistent PDMS thicknesses by spin-coating at about 500 RPM for about 15 seconds, followed by about 2000 RPM for about 20 seconds. A blank wafer 264 is similarly coated with a second layer of PDMS 269 and both are allowed to cure in an oven at about 60-65° C.

The cured PDMS from the pump channel master is then cut with a rotary razor (Olfa Manufacturing, 18 mm) as close to the channel edge as possible with the aid of a dissection microscope. The rectangular, ribbon-shaped section encompassing the channels is then carefully peeled up, inverted, placed on a glass slide (not shown), and it and the blank-wafer 211 with PDMS 216 (S4) are plasma-treated for about 20 seconds (Harrick Plasma Treater, PDC-32G) and bonded together such that channels 278 are completely encapsulated. The glass slide is then removed. Shown in FIG. 2(A), the encapsulated channels are then cut from the blank wafer 211 with the rotary razor according to preset boundaries, creating a very thin and narrow rectangular ribbon shaped piece 230 of PDMS (at steps S5 and S6). The encapsulated channel ribbon 230 is wrapped in a single helical turn around a blank tube 240 to create a helical wrap. The cam for this prototype can be a smashed piece of metal tubing, but a four-fluted reamer from McMaster-Carr of diameter 0.0500″ can also be used. The helical wrap is affixed to a substrate. A tape can hold the ends of both the channel ribbon and the rod to the bottom of a Petri dish. An assembly is then cast in the Petri dish in a thick layer of PDMS that provides support for the channels and a substance against which the channels can be compressed during the pumping motion. After the final casting has cured, the pump is cut out as a large block, peeled off the dish, and trimmed; the blank tubing is removed; and two tubing holes are punched to access the channels' inlet and outlet. Finally, the entire pump is plasma bonded to a 1×3″ or 2×3″ glass slide.

In the example, the channels are formed of PDMS. Other materials can also be utilized to practice the present invention. The materials are chemically biocompatible with a variety of cells including human T cells. Mechanical biocompatibility is inherent to the pump to reduce lysing (bursting) of cells wherein the action of the cam gradually compresses the fluid channel to forcing fluid along the flow direction. The gradual compression produce shear rates that are more tolerable than other types of pumps, such as a conventional pumps which provide a sudden compression instead of a gradual compression. This allows pumping of cellular solutions such as blood without lysing cells that pass through the pump.

In one embodiment, the pump is a positive displacement pumps discharge the same flow regardless of head pressure over the entire range of operation. The flow channel is completely occluded by a moving compression wave actuated by the lobes of the rotating cam. Such pump can typically pump against very large head pressures because there is no route of counterflow for the fluid. The long axis of the cam cross-section can be increased or decreased to apply more or less head pressure to the compression during the pump stroke. This is useful for pumping against larger head pressures. A careful design of the size of the long axis may reduce friction and mechanical wear on the PDMS channel structure, while provide sufficient head pressure. The pressure which causes the compression wave in the present invention is delivered by a substantially incompressible metal cam, therefore provide fast and linear response.

According to the present invention, the pumping exhibited no detectable backlash—reversal of the direction of rotation of the cam resulted in an immediate reversal of the direction of flow of the beads with no delay. Highly precise linear placement of the beads (1 micron beads in solution could be juxtaposed reliably to 1 micron beads stuck to the channel walls) may suggest that the flow control may be limited by the precision of the driving mechanism or motor, not by the pump itself.

In one embodiment, a smaller cam diameter, a cam diameter of 0.018-0.020″ is used. This generates a smaller stroke volume of the helical channels due to the reduced diameter of the cam, and targets a physiologically relevant range of flow rates for cellular microfluidic studies of 100-1000 nl/min. This very small diameter cam shaft poses some challenges to fabrication mostly because of its small size. This embodiment was used to connect the macro world to a multi-trap nanophysiometer by means of 50 μm internal diameter (ID) PEEK tubing. Using a polished, smashed piece of tubing a reliable flow with this device can be realized and noted very little friction during rotation of the cam. It was shown that it was possible to successfully pump 1 micron and 5 micron fluorescent microspheres from the inlet, through the pump, through the tubing and finally through the multi-trap nanophysiometer using human fingers to drive the cam rotation.

Several exemplary methods for fabricating pump cams are demonstrated, including at least a helical wrap of 25 μm wire around a 0.5 mm OD, 0.4 mm ID metal tube with an epoxy adhesive, off-the-shelf fluted reamers, and dental root canal files. The method for producing cams is to compress sections of thin-walled stainless steel tubing in a bench vise with smooth-surface square jaws. In this embodiment, a fairly precise vise is designed to be mounted on the horizontal bed of a vertical milling machine (Bridgeport) with smooth surfaces on the inside of the jaws. By orienting the tubing at 45° and placing it near the upper corner, approximate as much length as required for compression of the channels is deformed. This reduces the friction from cam rotation during operation and motor load. Compressing the tubing until it is significantly flat may result in a functioning cam. However, in another embodiment, cams are compressed less than completely flat by placing layers of shim stock in the vise to limit the final compressed thickness. Beginning with 0.5 mm OD tubing, a typical compression creates a cam with a cross-sectional ellipse having a major diameter of 0.67 mm and a minor diameter of 0.33 mm.

It is required that the cam be as smooth and free from debris as possible before introduction into the cam shaft since the PDMS may be damaged by metal filings or shards. There are a variety of polishing compounds that work with stainless steel. One can finish with Dremel No. 421 (Robert Bosch Tool Corporation), a silicon-carbide-based polishing compound which imparts a high luster to the stainless cam. The cam can be rotated at high speed with a rotary tool to increase the speed of polishing, and it should be periodically inspected during polishing and compared with an unpolished tube under a microscope until it has a mirror-like finish. It is important in many of the embodiments to remove debris, including dust, skin flakes, and metal filings, that finds its way between the cam and the PDMS of the pump, as any that remains may decrease the life of the pump.

In one embodiment, the flow can be controlled and measured. Although flow generated by the pump is obvious to the operator by the droplets of media that quickly form at the outlet of the pump device or tubing, or by the microscopic observation of moving cells, beads, or debris in downstream MF devices, it was found that measurement of absolute and instantaneous flow is difficult. Longterm average flow measurements were made by connection of an inline flow meter with 5% accuracy (Upchurch, Nano Flow) controlled by, for example without limitation, a LabView program, a Visual Basic interfacing program or a Visual C program. The flow meter uses thermal anemometry to measure flow in the range of 1.5 nL/min to 8.0 μL/min, but is sensitive to particulate matter in the fluid. One can also estimate flow by pumping fluid into small-bore transparent tubing (Cole-Parmer P/N EW-06418-02) with an ID of 0.5 mm and measuring the travel of the fluid-air interface over time with a set of machinist's digital calipers. The tubing has a volume of approximately 200 nanoliters per millimeter of length. Five to fifteen minutes of flow caused consistent displacements of over ten millimeters which could be measured with good accuracy and precision with the calipers. Neither of these methods was able to provide instantaneous flow measurements since the flow meter is limited to DI water and has an update rate of 2 Hz, and the standpipe/tubing method requires several minutes to detect a change in the meniscus position. For instantaneous flow detection, a particle image velocimetry (PIV) that runs as, as an example without limitation, an ImageJ plug-in PIV combined with simple visual inspection of cells, beads, or debris provided a satisfactory method of testing the effectiveness of different cams and pumps. Particle velocity in downstream devices as visualized through the eyepieces of a microscope exhibits substantially instantaneous response to cam rotation either by hand or by a motor.

In one embodiment, a nanopumps with pancake-style stepper motors was used. Miniature DC slot-car motors, and spring drives from mechanical toys, and all work more or less equally well as a source of steady-state motion, they may lack the combined ability to produce adequate torque at various velocities, encode the positions, and rotate a predetermined metered distance in a compact package. Furthermore, the speed of some motors, such as the DC motors, can fluctuate slightly over long time periods. A 2-stage miniature stepper motor (MicroMo Electronics, ADM_{—}0620) using a simple USB stepper motor controller (Allmotion, EZ10EN), in the present context, for driving the pumps provides alternative option. The motor body is approximately 6 mm diameter and 9 mm in length, has a 1 mm diameter shaft, and may be equipped with an optional 3.3 mm gear head and encoder. It is capable of a range of rotational velocities and can also operate in step mode, rotating a commanded amount and stopping, thus causing the pump to dispense metered amounts of fluid that are a fraction of the stroke volume of the pump. The optional encoder may ensure that the commanded rotational angle is achieved without failure. The motor has twenty full steps per turn and can be micro-stepped to achieve very small rotational angles by further divide the full steps into smaller steps. In the micro-stepped mode, the theoretical limit of fluid dispensing is about 150 pL per step. The controller software is simple to install, and custom motor communication software is not difficult to produce. The controller used has a footprint approximately one half the size of a credit card, and an even smaller motor controller using surface mount devices with tight lead spacing to achieve very compact controller-motor housing is developed. At present, a single miniature motor, gear head, encoder, and controller cost is reasonable.

The friction may degrade the performance of the pump with sustained operation. It was found that with long delays in which the pump was dried and allowed to sit, it still performed well when primed and operated. A stepper motor was connected to the cam and set the pump at approximately one hundred revolutions per minute and let it run for three days with no loss of function. Although the pump ran dry during this endurance test, the pump channels did not lose the integrity and the pump was still able to perform when primed. After several similar tests it was concluded that the pump structure, including at least the channels, was capable of sustained operation.

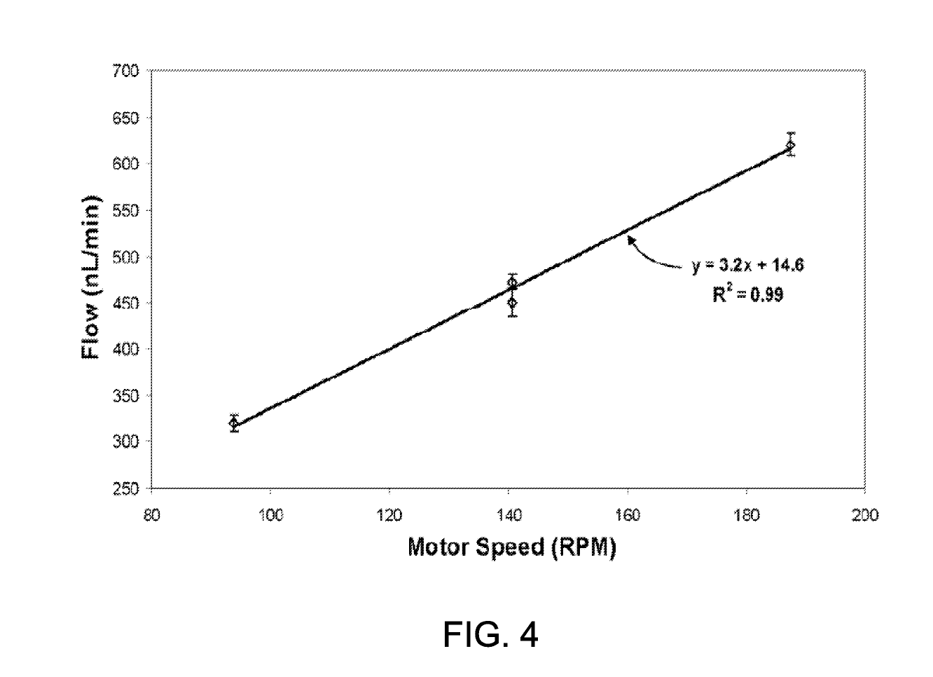

The consistency of the pumping rate over time is evaluated in a pump according to one embodiment of the present invention. In this evaluation, the pump runs over several hours at three different motor speeds, resulting in a positive linear correlation of flow rate and RPM and an actual stroke volume of 3.2 nL. Data are shown in FIG. 4, where a linear fit to the data shows that the stroke volume for this pump is 3.2 nL, which is in good agreement with a predicted value of 5.4 nL if a compression fraction of 40% is assumed. Data points are the averages of four measurements at each motor RPM made every 15 minutes. Error bars are the standard deviation. An a priori calculation of stroke volume for ten channels of 11×25 μm cross-section in a helical conformation without compression is 5.4 nl. the compression of 40% explains the discrepancy between theoretical and actual stroke volumes.

To visualize compression caused by the cam one can imagine a clock face overlaying the cross-section of the pump with two lobes of the cam compressing channels that span about twelve minutes on opposite sides of the clock simultaneously. A prior test with a different motor yielded a less consistent correlation of flow rate with motor RPM. The inconsistency may be caused by plugging or clogging of any of the ten parallel channels, which is certain to significantly affect stroke volume. Greater caution was executed at testing the consistency of the pump, as shown in FIG. 4.

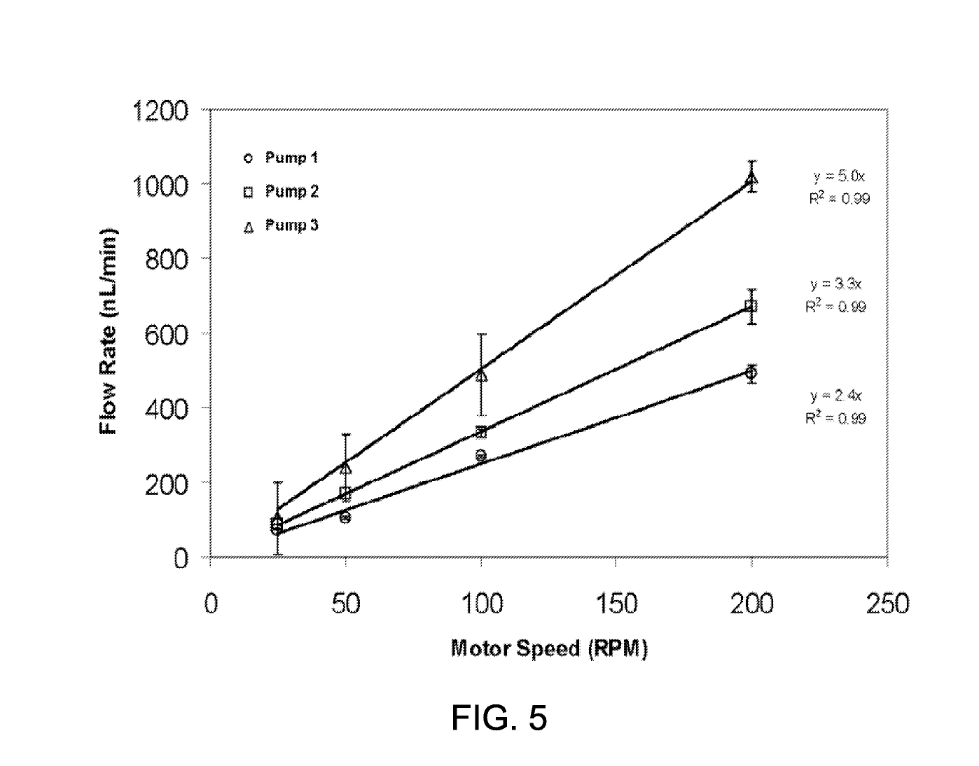

To test pump consistency across different pumps, three pumps were manufactured and tested them with the same cam and stepper motor at four different speeds, while estimating the flow with the tubing water-air interface method. FIG. 5 shows flow rates plotted versus motor RPM for the three pumps. The stroke volumes measured on this graph vary from 2.5 nL for Pump 1 to just over 5 nL for Pump 3 (linear fits not shown). An unexpected difference in stroke volume, possibly due to occlusion of one or more sub-channels, explains the discrepancy between pumps.

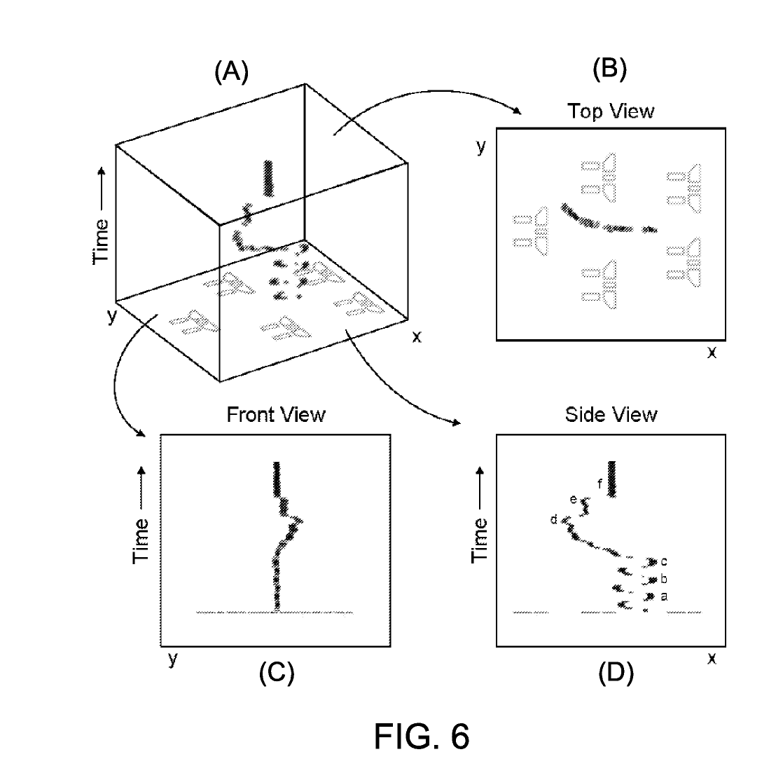

According to the present invention, a fast response time in downstream devices to the operation of the cam can be achieved. As shown in FIG. 3, the cam 308 can be operated by hand. Tube 302 is filled with water containing a low concentration (1-5%) of 1 μm polystyrene beads, and device 350 was a multi-trap nanophysiometer mounted on a microscope stage. In this setup, microscopic visualization of bead motion in the downstream device provides instantaneous feedback on the effect of cam rotation on fluid velocity. FIG. 6 is a kymograph display that illustrates a segment of screen capture video approximately 20 seconds long featuring four microfluidic cell traps, with time increasing in the upward direction, where a pair of beads in a trap device are driven by a hand operated rotary nanopump. FIG. 6 shows the perspective (A) and top (B), front (C) and side (D) views. Time increases with height in the perspective drawing. With sequential reversals of direction caused by 8 turns of the cam in alternating directions, the bead completes 3 complete short round trips traveling in the vicinity of the trap before coming to rest. Trap baskets are approximately 18×18 μm². The tracing is the location of a coupled pair of about 1 μm polystyrene beads relative to a static reference point. Clockwise rotation of the cam one-quarter turn moves the beads about 20 μm forward in the trap device, and counterclockwise rotation of the cam by one quarter of a turn repositions the beads substantially close to the spot where the beads started, which is marked by “a” in the lower right panel (D) of FIG. 6. In present context, two more sequences of rotations followed by counter rotations produce substantially the same response in the bead, leaving it in its the substantially same XY location after three round trips, marked by “b” and “c”, respectively. Then a larger (approximately a half turn) counterclockwise rotation sends the beads in the negative X direction approximately 114 μm and positive Y direction approximately 37 μm, marked by “d”. The moving in the Y direction is dictated by the flow streams in the device which are determined by the configuration of traps in the device. Final clockwise rotations, marked by “e” and “f”, move the beads closer to the starting position, after which time the pump is no longer rotated and the beads remain stationary. A view of the kymograph from positive time looking down onto the XY plane, as shown in the top right panel (B) of FIG. 6, shows that the beads' trajectories overlay each other perfectly, substantially agreed with laminar, low Reynolds number flow. It is important to note that the round trip one quarter-turn trajectories each took less than 2 seconds to complete, and this was a casual rate of cam operation. This illustrates the instantaneous response of the MF fluid velocity to cam rotations, which is contemplated as a beneficial for good control of MF operations.

Similar studies have been conducted with different trap devices, different size beads, whole blood (diluted and undiluted), yeast, Jurkat cells, and human T cells, all with similar results. In one embodiment, the head pressure limit of the pump is determined. A pressure bomb uses stainless fittings in order to receive pressure from a standard two-stage high pressure nitrogen gas regulator and provide constant head pressures between 0 and 100 kPa (0 and 15 PSI). PEEK fittings (Upchurch) can be used to couple Cole-Parmer 0.5 mm ID tubing from the outlet of the pump to the pressure bomb. By reading the distance traveled by the liquid/air interface, a measure of flow at different head pressures could be obtained.

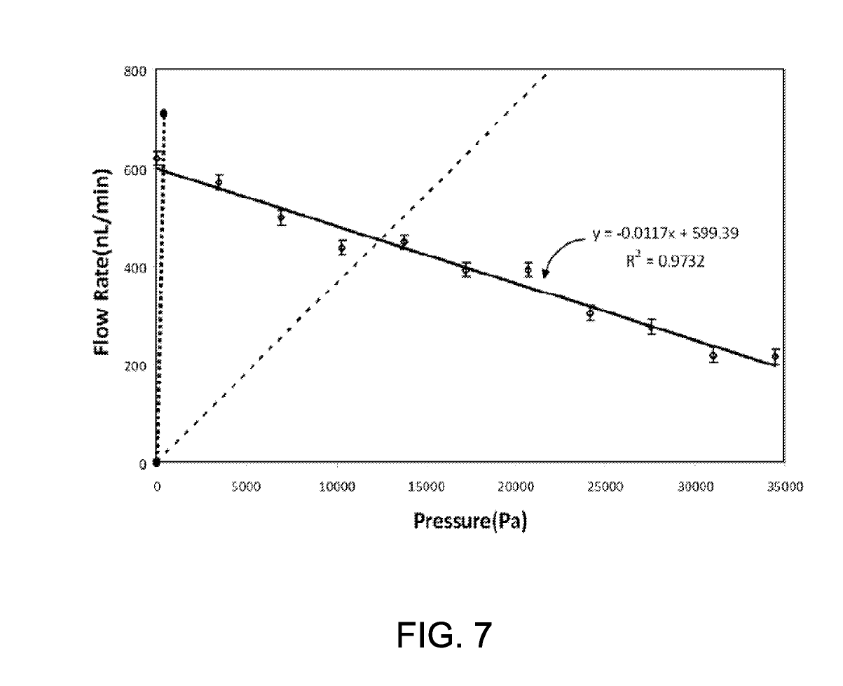

FIG. 7 illustrates the results at head pressure values from 0 to 33 kPa PSI at a motor RPM near the target flow of about 500 nL/min (at 187 RPM). Dashed line indicates the Hagen-Poiseuille relation for 25 cm of 50 μm ID cylindrical tubing—a typical connection length of PEEK tubing. Dotted line indicates the perfusion pressure of a complex, multi-cellular nanobioreactor with cells. The flow rate of the pump decreases linearly up to approximately 35 kPa. Visual confirmation with other pumps in the same setup indicates that the fluid begins to flow backward through the pump at or around 30 kPa, possibly representing a limit to this type of pump set by the elastic moduli of PDMS.

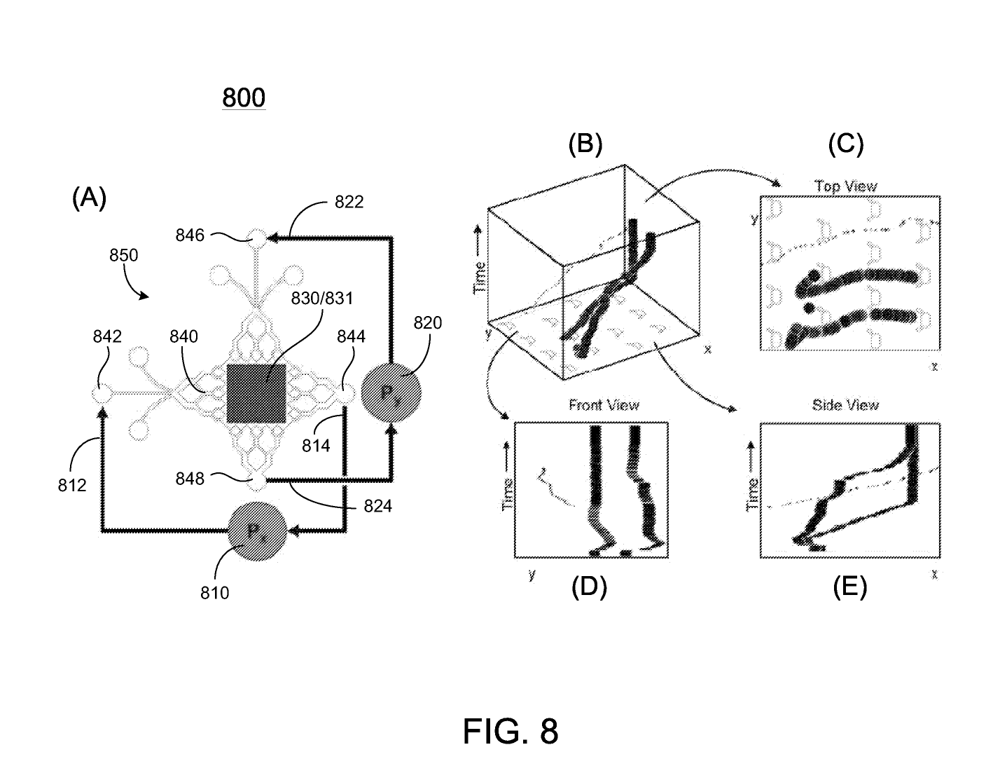

To estimate the flow rate when connected to a load, one can calculate the Hagen-Poiseuille flow versus pressure relationship for an idealized 30 cm section of tubing with a 50 μm inner diameter (dashed line), and the measured pressure versus flow relationship for a MF bioreactor. For the target flow of 500 nL/min, at a motor velocity of approximately 200 RPM the positive displacement pump performs well with a typical microfluidic device. The lead tubing of 30 cm provides actual resistance the pump is likely to encounter for typical cell-based microfluidic experiments. This means that for the typical MF applications, the pump operates closer to the Y-axis of FIG. 8 than the dashed line, so that it can first order be treated as a high-impedance, constant-volume flow source whose rate is determined by the motor speed rather than the head pressure at the head output.

As shown in FIG. 7, the pressure-response of the pump at one motor speed is illustrated. The Y-intercept may scale with different motor RPM values. For example without limitation, a motor RPM of 400 produce close to 1000 nL/min flow at minimum head pressure. For devices with resistance profiles comparable to the solid line trace, the flow rates are substantially limited by the RPM of the motor for which an upper limit has not been identified. The maximum head pressure may change with motor speed, but since the flow regimes are dominated by viscous forces it is safe to assume it remains near 5 PSI as it is in the plotted data. One can thus begin to picture the plots of pump performance at different motor speeds, head pressures, and device resistances. A stiffer PDMS against which the channels are compressed or slightly wider cams or both may have a positive effect on the maximum achievable head pressure.

According to the present invention, the precision with which velocity within the device can be controlled with the nanopump suggests the possibility for positioning, dosing, and manipulation of cells and groups of cells by using at least a first pump and a second pump to drive orthogonal flow streams in a downstream device.

Referring now to FIG. 8(A), a system 800 for manipulation of cells is shown according to one embodiment of the present invention. In one embodiment, the system 800 includes two pumps attached to the inputs and output of a downstream device to create perpendicular flow streams inside a square trap chamber. Specifically, the downstream device 850 has a plurality of cell traps 830 arranged in a square trap chamber defining a trap region 831 thereof, and a network of binary flow splitters 840. For example, in one embodiment, 1974 cell traps are arranged in the square chamber 831. Each face of the trap region 831 is connected to the network of binary flow splitters that divide or gather the flow uniformly across the entire side. The network of binary flow splitters 840 has a first input 842 and a first output 844 along the x direction and a second input 846 and a second output 848 along the y direction perpendicular to the x direction. As connected, the inlet 812 and the outlet 814 of a first pump Px 810 are respectively in fluid communications with the first input 842 and the first output 844 of the network of binary flow splitters 840, while the inlet 822 and the outlet 824 of a second pump Py 820 are respectively in fluid communications with the second input 846 and the second output 848 of the network of binary flow splitters 840.

The connection of the first input 842 and the first output 844 of the network of binary flow splitters 840 to the inlet 812 and the outlet 814 of a first pump Px 810 leads to flow streams, in the absence of traps, that would be substantially in the x direction, whereas the connection of the second input 846 and the second output 848 of the network of binary flow splitters 840 to the inlet 822 and the outlet 824 of a second pump Py 820 creates vertical flow streams. When one pump is operating, the flow streams are due substantially to that pump, but when both pumps are running the flow streams are the vector addition of the individual components. This allows for the positioning and movement of single cells anywhere within the boundaries of and on the two-dimensional region defined by the square trap chamber. This aspect of present invention may be very useful for cell interaction experiments such as cell-cell signaling or selective multi-cellular fusion. In practice, the traps and other features (such as bubbles and cells) obstruct and divert the flow streams, such that the flow lines from each pump wherein the flow lines from each pump may be substantial aligned with the x and y axes, and the two axes are orthogonal to each other. In the present context, the high-impedance of the nanopump ensures that the net flux across opposite faces is zero. Consistent with this, a streamline exiting one pump might enter the other.

FIGS. 8(B)-8(E) are kymograph plots showing 2-D flow control in a perspective view (B), a top view (C), a front view (D) and a side view (E), performed with the cross-flow device 800, which shows controllably repositioning of two cells from the locations where the cells were originally trapped to two other traps in different rows of the trap field using hand-driven pumps. Cells in adjacent traps are repositioned in about 12 seconds to adjacent traps two columns to the right using hand-driven pumps and a setup similar to the top panel, where the two cells are driven out of the traps in the first 12 seconds due to the action of one of the pumps, and then steered into two adjacent traps two rows behind the original traps by the operation of the other pump. According to the present invention, the ability of the two nanopumps to decompose the device flow streamlines into individual, independently controllable components enables the manipulation of the fluid mass.

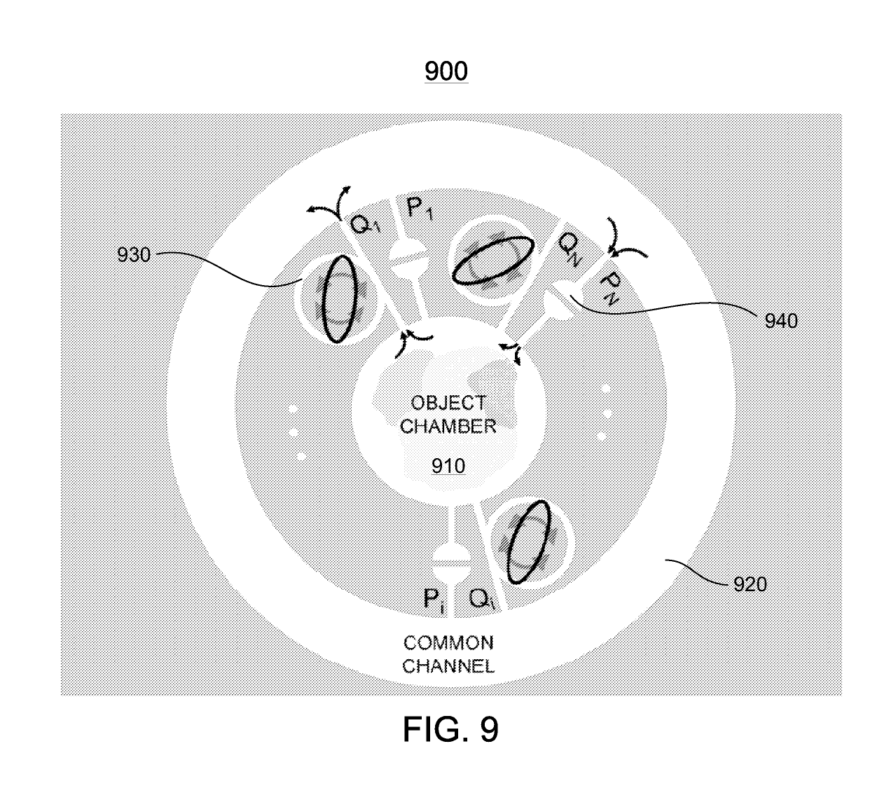

In one embodiment, an obvious extension of the two-pump orthogonal flow controller of FIG. 8 is an N pump system for fluidic impedance tomography (FIT), as shown in FIG. 9. In this embodiment, the FIT system 900 includes an object chamber 910, a common channel 920 spaced-apart surrounding the object chamber, N pumps 930 spaced-apart coupling between the object chamber 910 and the common channel 920 and N pressure transducers 940, each pressure transducer 940 connected between the object chamber 910 and the surrounding common channel 920 and associated with a respective pumps 930 for determining the pressure difference between the periphery of the object chamber 910 and the surrounding common channel 920 therein. The measurement of the pressure distribution P, for each of a series of flow Q, distributions is used to reconstruct a fluidic impedance distribution within the object chamber.

The pattern of flow through the central chamber 910 is determined by the rate and direction of the flow Qᵢ driven by each pump, where 1≦i≦N, with the constraint

$${\sum }_{i = 1}^{N} {Q}_{i} = 0$$

that if the fluid is incompressible and the channels are non-distensible. In other word, the sum of the pump flows between the sample chamber and a common outer channel 920 sum to zero. If desired, microfabricated pressure transducers, for example without limitation, using optical measurements of the displacement between thin membranes, could determine the pressure difference between the periphery of the central chamber and the surrounding common channel. The pattern of flows that might be achieved in the central chamber is limited by hydrodynamic constraints—in the quasistatic limit where inertial effects can be ignored, the flow patterns may be determined by the gradient of the pressure distribution, which in turn satisfy Laplace's equation with the boundary conditions associated with the injection or removal from the fluid and the distribution of fluidic impedance associated with the objects contained in the central chamber. Noting the parallel with electrical impedance tomography (EIT) in which differing distributions of electrical current are injected and removed at the edge of a planar object and the resulting potential distributions are measured along the periphery to determine the electrical impedance distribution within the object [25-27], the measurement of the pressure distribution P, for each of a series of different Q′ distributions is used to reconstruct the fluidic impedance distribution within the central chamber 910, i.e., fluidic impedance tomography (FIT). In one embodiment, the surrounding common channel has a channel depth adapted such that a fluidic impedance of the surrounding common channel is negligible relative to that of the object chamber. The N pumps are high-impedance flow sources. The N pressure transducers are stiff. Particle velocimetry is often used to image directly the flow velocities. FIT is applicable in those applications with opaque or scattering fluids or hydrogels. The mathematics developed for EIT is adaptable for the determination of the Laplace-related constraints on the complexity of the flow patterns that is established using only peripheral injection or withdrawal of fluid, for example, to allow the forward determination of the fluid flow distribution that optimizes the delivery of collections of cells to specific traps such as those shown in FIG. 8.

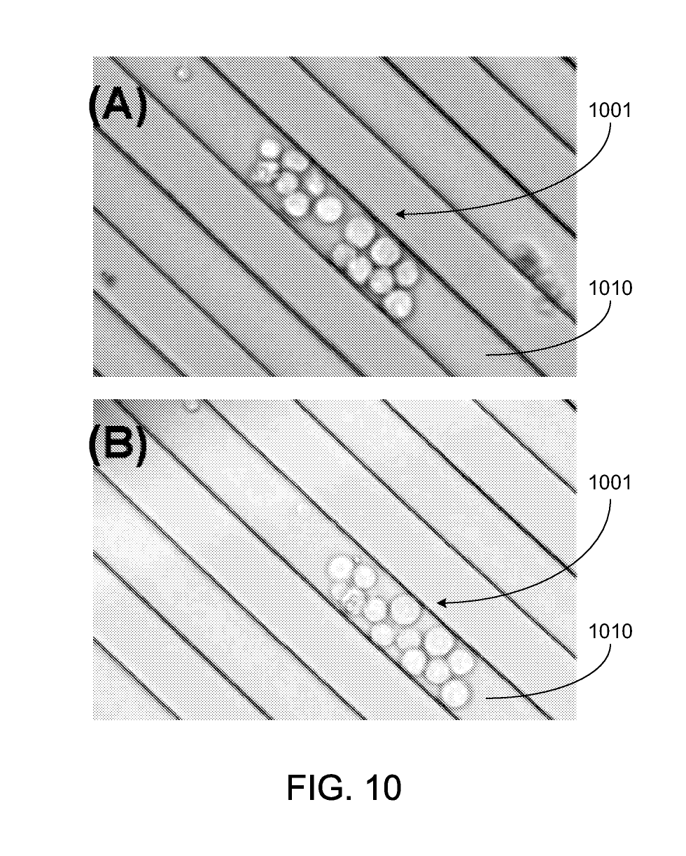

Cell visibility in microfluidic devices is considerable concern for many researchers. According to the present invention, the system 900 offers the researcher an opportunity to exercise very tight control over the microenvironment of the cell for extended time periods, but the opportunity comes at the cost of having to decide and determine the many variables that can affect cell fate, and to maintain the vigilance that the environment is maintained within preset limits. Although any cell can suddenly cease to be alive through a host of factors leading to necrosis, cells can also respond to many different cues in the environment by initiating apoptosis, or programmed cell death, which is a process that can take hours to complete. Therefore, isolation of one factor and its causative effect on cell viability can be a very cumbersome, if not impossible, chore. Nonetheless, one is interested in the effect of the pump on the health and viability of cells that transit it. It is observed that many types of cells, including human T cells, Jurkat human T cell line, diluted whole blood, and yeast, that have successfully transited the pump and arrived in the downstream device with no apparent ill effect. FIG. 10 illustrates a cluster of Jurkat T cells (each 6-10 μm in diameter) in a pump channel, where FIG. 9(A) shows a cluster of 14 cells 1001 upstream of the pump moving down and to the right, and FIG. 9(B) shows the same cluster of cells 1001 downstream of the pump after being driven through the helix channels 1010 by the traveling compression wave, still moving down and to the right. The orientation of the cells 1001 with respect to each other appears slightly changed, indicating that the cluster did not experience excessive shear forces during transit. This cluster transited the pump significantly out of phase with the compression zones caused by the rotating pump cam, and thus the cells were not damaged. However, even if the cells were collected near the sweep of the cam lobes, it is substantial less likely that the cells may have been “pinched” or compressed by the compression zone. While careful observation of the pump during operation with fluorescent beads over many experiments does indicate that some beads tend to collect in the pump channel, there are never a significant number of damaged or lysed cells in the downstream devices in any experiments with cell solutions pumped at moderate concentrations. This may be the laminar flow within the periodically compressed microchannels.

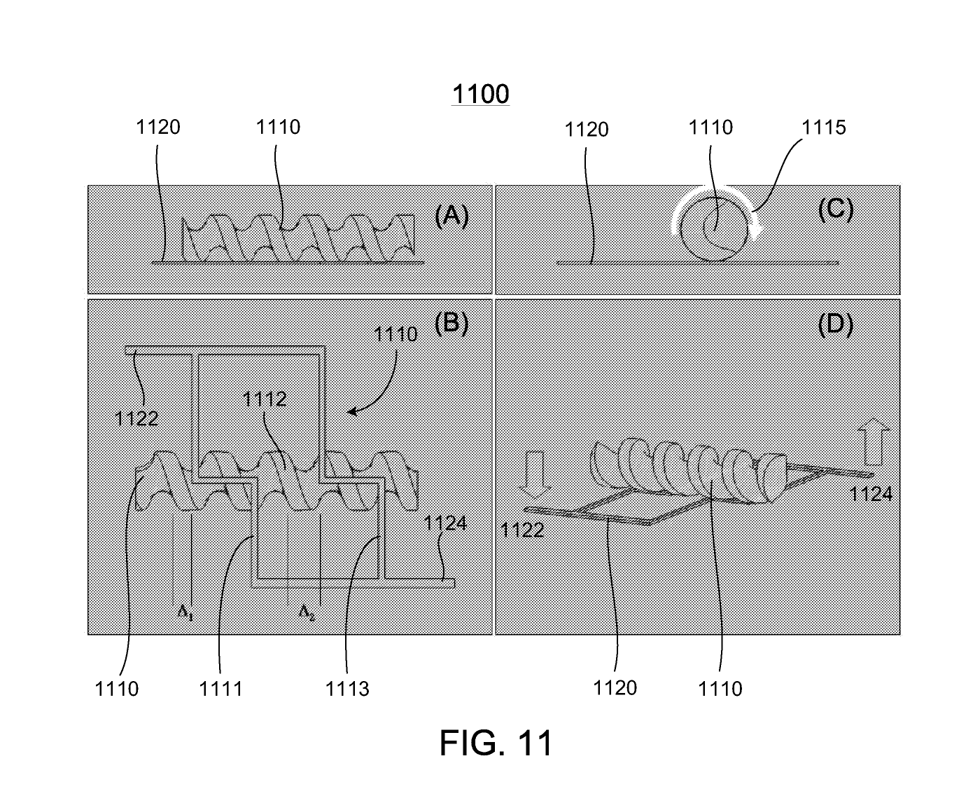

In one aspect, the present invention relates to a pump having planar fluidic channels, which are relatively easy to fabricate. As shown in FIG. 11, the pump 1100 has a network of linear fluidic flow channels 1120 that diverge from an outlet and converge at an inlet, disposed on a substrate; and an external threaded rod 1110, and rotatably placed on the network of linear fluidic flow channels 1120, such that when the threaded rod 1110 rotates, the ridge 1112 of the threaded rod 1110 compresses one or more of the fluidic flow channels 1120 to produce a wave of compression that actuates a peristaltic flow of fluid therein so as to direct a desired amount of the fluid toward one of the inlet 1122 and the outlet 1124. The threaded rod 1110 is in an Acme thread form or a trapezoidal form.

In operation, the helical threaded rod 1110 compress a network of linear microfluidic flow channels 1120 comprising multiple channels that diverge from an inlet 1122 and converge at an outlet 1124 (Arrows in FIG. 11(D)). For simplicity, two channels are depicted in the embodiment. Rotation of the rod, as shown in FIG. 11(C), causes a wave of compression that drives flow through the microfluidic network 1120. FIGS. 11(A)-11(D) shows respectively side, bottom, front and perspective views of the pump 1100. There are the different offsets Δ₁ and Δ₂ of the first and second channels relative to the repeating helical structure of the threaded rod 1110, as shown in FIG. 11(B). The function of the phased offsets, in one embodiment, is to reduce flow pulsatility in the main outlet of the pump. While each compression zone of the fluidic channel spans one thread, channels which span more than one thread are possible. The diagonal arrows indicate compression of the microfluidic channel by the threads, and the horizontal ones show the width of the channel. The rotation moves the compression wave along the direction of flow. The planar pump has an assembly that is simple. The head pressure, stroke volume, and flow rates of this pump depend on the type of threads and their interaction with the channels of PDMS. Since the RPM of a motor can be adjusted over a wide range, the pump can deliver an equally wide range of flow rates. The metering capability is preserved because rotation of the motor by a fixed number of turns still dispenses a fixed increment of fluid.

According to the present invention, the planar pumps has mechanical biocompatibility, reduced pulsatility, and simple driving mechanisms. A silicon master for this prototype is made and the PDMS microfabrication is similar to the helical wrapping. The coupling of the rod to the PDMS channels with this prototype may be more complicated, but may ultimately be conducive to high-throughput manufacturing methods.

In one aspect of the present invention, a method for supplying continuous fluid flow to microfluidic devices is disclosed. A series of parallel channels are helically wrapped around a cam shaft, in which a non-cylindrical cam is rotated, compressing the channels in traveling waves to create fluid flow. Theoretical calculations compared to experimental data confirm the effectiveness of the pump design. The pump has been demonstrated to produce flow rates from below 50 nL/min to above 1 μL/min with a stroke volume of 3-5 nanoliters per revolution against head pressures of up to 5 PSI. Lower and higher flow rates are possible with different sizes and numbers of microfluidic channels. The pump can be interconnected with microfluidic devices using smallbore PEEK tubing, and the possibility of direct integration as a component of many types of microfluidic devices suggests that the pump design may be a valuable tool for point of care diagnostics.

In one embodiment, the pump can be integrated with a bank of Quake-style valves and the multi-trap nanophysiometer. For example, the integrated device can be used to sample finger-stick volumes of blood and separate the leukocytes from the red cells (in the nanophysiometer), load the cells with calcium dye, collect calcium transients in response to various important chemical stimuli and sequentially label the trapped leukocytes with CD markers to determine their types (B lymphocytes, T lymphocytes, neutrophils, etc). The present embodiment in current context may be useful clinically both in developed and underdeveloped areas.

The present invention, among other things, discloses the design, fabrication, and testing of a microfabricated metering rotary nanopump for the purpose of driving fluid flow in microfluidic (MF) devices. The miniature peristaltic pump includes a set of microfluidic flow channels wrapped in a helix around a central cam shaft in which a non-cylindrical cam rotates. When rotating, the cam shaft compresses the helical channels to induce a peristaltic flow. The nanopump is able to produce intermittent delivery or removal of several nanoliters of fluid per revolution as well as consistent continuous flow rates ranging from as low as from about 15 nL/min to above 1.0 μL/min. At back pressures encountered in typical MF devices, the pump acts as a high impedance flow source. The durability, biocompatibility, ease of integration with soft-lithographic fabrication, the use of a simple rotary motor instead of multiple synchronized pneumatic or mechanical actuators, and the absence of power consumption or fluidic conductance in the resting state all contribute to a compact pump with a low cost of fabrication and versatile In one embodiment, the channels are formed of PDMS implementation. This suggests that the pump design may be useful for a wide variety of biological experiments and point of care devices, such as the fluid dynamics of MEMS devices with precise control of multiple flow inputs.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### REFERENCE LIST

[1]. West, J., Becker, M., Tombrink, S. & Manz, A. Micro total analysis systems: Latest achievements. Analytical Chemistry 80, 4403-4419 (2008).
[2]. Chang, S. T., Beaumont, E., Petsev, D. N. & Velev, O. D. Remotely powered distributed microfluidic pumps and mixers based on miniature diodes. Lab on a Chip 8, 117-124 (2008).
[3]. Wikswo, J. P. et al. Engineering challenges of BioNEMS: the integration of microfluidics, and micro- and nanodevices, models, and external control for systems biology. IEE Proc.-Nanobiotechnol. 153, 81-101 (2006).
[4]. Werdich, A. et al. A Microfluidic Device to Confine a Single Cardiac Myocyte in a Sub-nanoliter Volume on Planar Microelectrodes for Extracellular Potential Recordings. Lab Chip 4, 357-362 (2004).
[5]. Faley, S. et al. Microfluidic platform for real-time signaling analysis of multiple single T cells in parallel. Lab on a Chip 8, 1700-1712 (2008).
[6]. Faley, S. L. et al. Microfluidic single cell arrays to interrogate signalling dynamics of individual, patient-derived hematopoietic stem cells. Lab on a Chip 9, (2009).
[7]. Prokop, A. et al. NanoLiterBioReactor: Long-Term Mammalian Cell Culture at Nanofabricated Scale. Biomed. Microdevices 6, 325-339 (2004).
[8]. Seale, K. T., Faley, S., Chamberlain, J. & Wikswo, J. Macro to nano: A simple method for transporting cultured cells from milliliter scale to nanoliter scale. Exp. Biol. Med. (2010).
[9]. Cheng, X. H. et al. A microchip approach for practical label-free CD4+ T-cell counting of HIV-infected subjects in resource-poor settings. Jaids-Journal of Acquired Immune Deficiency Syndromes 45, 257-261 (2007).
[10]. Huang, C. J., Chen, Y. H., Wang, C. H., Chou, T. C. & Lee, G. B. Integrated microfluidic systems for automatic glucose sensing and insulin injection. Sensors and Actuators B-Chemical 122, 461-468 (2007).
[11]. Laser, D. J. A review of micropumps. Journal of Micromechanics and Microengineering 14(6), R35-R64. 2004. Ref Type: Abstract.
[12]. Namasivayam, V., Larson, R. G., Burke, D. T. & Burns, M. A. Transpiration-based micropump for delivering continuous ultra-low flow rates. Journal of Micromechanics and Microengineering 13, 261-271 (2003).
[13]. Manz, A. et al. Electroosmotic Pumping And Electrophoretic Separations For Miniaturized Chemical-Analysis Systems. Journal of Micromechanics and Microengineering 4, 257-265 (1994).
[14]. Ross, D., Johnson, T. J. & Locascio, L. E. Imaging of electroosmotic flow in plastic microchannels. Analytical Chemistry 73, 2509-2515 (2001).
[15]. Studer, V., Pepin, A., Chen, Y. & Ajdari, A. An integrated AC electrokinetic pump in a microfluidic loop for fast and tunable flow control. Analyst 129, 944-949 (2004).
[16]. Ma, H. K. et al. Development and application of a diaphragm micro-pump with piezoelectric device. Microsystem Technologies-Micro- and Nanosystems-Information Storage and Processing Systems 14, 1001-1007 (2008).
[17]. Unger, M. A., Chou, H. P., Thorsen, T., Scherer, A. & Quake, S. R. Monolithic microfabricated valves and pumps by multilayer soft lithography. Science 288, 113-116 (2000).
[18]. Gu, W., Zhu, X., Futai, N., Cho, B. S. & Takayama, S. Computerized microfluidic cell culture using elastomeric channels and Braille displays. PNAS 101, 15861-15866 (2004).
[19]. Faley, S. L. et al. Microfluidic single cell arrays to interrogate signalling dynamics of individual, patient-derived hematopoietic stem cells. Lab Chip 9, 2659-2664 (2009).
[20]. Wlodkowic, D., Faley, S., Zagnoni, M., Wikswo, J. P. & Cooper, J. M. Microfluidic single-cell array cytometry for the analysis of tumour apoptosis. Anal. Chem. 81, 5517-5523 (2009).
[21]. Faley, S. et al. Microfluidic platform for real-time signaling analysis of multiple single T cells in parallel. Lab Chip 8, 1700-1712 (2008).
[22]. Melin, J. & Quake, S. R. Microfluidic large-scale integration: The evolution of design rules for biological automation. Annual Review of Biophysics and Biomolecular Structure 36, 213-231 (2007).
[23]. Wang, X. Y., Cheng, C., Wang, S. L. & Liu, S. R. Electroosmotic pumps and their applications in microfluidic systems. 6, 145-162 (2009).
[24]. Raffael, M., Willert, C. & Kompenhans, J. Particle Image Velocimetry: A Practical Guide. Springer-Verlag, Berlin (2007).
[25]. Borcea, L. Electrical impedance tomography. Inverse Problems 18(6), R99-R136. 2002. Ref Type: Abstract.
[26]. Frerichs, I. Electrical impedance tomography (EIT) in applications related to lung and ventilation: a review of experimental and clinical activities. Physiol. Meas. 21, R1-R21 (2000).
[27]. Brown, B. H. Medical impedance tomography and process impedance tomography: a brief review. Meas. Sci. Technol. 12, 991-996 (2001).

What is claimed is:

1. A pump, comprising:
(a) a cam having a cam shaft, wherein the cam shaft has an axis, an exterior surface and M fins spaced-apart formed on the exterior surface along the axis; and
(b) at least one fluidic flow channel having an inlet and an outlet, disposed on a substrate, wherein the at least one fluidic flow channel helically surrounds the cam shaft such that when the cam shaft driven by the cam rotates, the M fins of the cam shaft compresses the at least one fluidic flow channels to produce a wave of compression that actuates a peristaltic flow of fluid therein so as to direct a desired amount of the fluid toward one of the inlet and the outlet.

2. The pump of claim 1, wherein the cam has a non-circular cross-section.

3. The pump of claim 1, wherein the M fins are parallely formed on the exterior surface along the axis.

4. The pump of claim 3, wherein the M fins are helically formed on the exterior surface along the axis.

5. The pump of claim 1, wherein the at least one fluidic flow channel comprises a plurality of fluidic flow channels defining a network of channels that converges at one of the inlet and the outlet and diverges at the other of the inlet and the outlet.

6. The pump of claim 1, wherein the at least one fluidic flow channel is biocompatible.

7. The pump of claim 6, wherein the at least one fluidic flow channel is formed of polydimethylsiloxane (PDMS).

8. The pump of claim 1, wherein the at least one fluidic flow channel has a cross section in a geometric shape of a circle or a polygon.

9. The pump of claim 8, wherein the cross section has a maximal dimension in a range of about 1-100 μm.

10. The pump of claim 1, wherein the rotation of the cam shaft is reversible, wherein when the cam shaft rotates in a reversed direction, the desired amount of the fluid is directed toward the other of the inlet and the outlet.

11. The pump of claim 1, wherein when the cam shaft rotates to position one of the M fins substantially against the substrate, the fin compresses the at least one fluidic flow channel to interrupt the flow of the fluid therein, thereby producing the flow of the fluid with M interruptions per revolution.

12. The pump of claim 11, wherein the produced flow of the fluid is of a pulsatile flow.

13. The pump of claim 1, wherein in operation, the cam shaft rotates at a constant speed or a variable speed.

14. The pump of claim 1, wherein the flow rate of the fluid is controllable by at least one of the size and the number of the fluidic flow channels and the rotating speed of the cam shaft.

15. The pump of claim 1, further comprising means for rotating the cam.

16. A pump, comprising:
(a) a network of linear fluidic flow channels that diverge from an outlet and converge at an inlet, disposed on a substrate; and
(b) an external threaded rod, and rotatably placed on the network of linear fluidic flow channels, such that when the threaded rod rotates, the ridge of the threaded rod compresses one or more of the fluidic flow channels to produce a wave of compression that actuates a peristaltic flow of fluid therein so as to direct a desired amount of the fluid toward one of the inlet and the outlet.

17. The pump of claim 16, wherein the network of linear fluidic flow channels is biocompatible.

18. The pump of claim 17, wherein the network of linear fluidic flow channels is formed of polydimethylsiloxane (PDMS).

19. The pump of claim 16, wherein each fluidic flow channels has a cross section in a geometric shape of a circle or a polygon.

20. The pump of claim 19, wherein the cross section has a maximal dimension in a range of about 1-100 μm.

21. The pump of claim 15, wherein the flow rate of the fluid is controllable by at least one of the size and the number of the linear fluidic flow channels and the pitch and the ridge width and the rotating speed of the threaded rod.

22. The pump of claim 16, wherein each fluidic flow channel has a corresponding offset relative to the repeating helical structure of the threaded rod, wherein the offsets of the fluidic flow channels are different from one another, thereby creating phased offsets of the flow from the network.

23. The pump of claim 16, wherein the rotation of the threaded rod is reversible, wherein when the threaded rod rotates in a reversed direction, the desired amount of the fluid is directed toward the other of the inlet and the outlet.

24. The pump of claim 16, wherein the threaded rod is in an Acme thread form or a trapezoidal form.

25. A pump, comprising:
(a) at least one fluidic flow channel having an inlet and an outlet; and
(b) means rotatably engaged with the at least one fluidic flow channel for periodically producing a wave of compression in the at least one fluidic flow channel, wherein the wave of compression actuates a peristaltic flow of fluid in the at least one fluidic flow channel so as to direct a desired amount of the fluid toward one of the inlet and the outlet.

26. The pump of claim 25, wherein the means comprises an external threaded rod such that when the threaded rod rotates, the ridge of the threaded rod compresses the at least one fluidic flow channel to produce the wave of compression.

27. The pump of claim 25, wherein the means a cam having a cam shaft, wherein the cam shaft has an axis, an exterior surface and M fins spaced-apart formed on the exterior surface along the axis such that when the cam shaft driven by the cam rotates, the M fins of the cam shaft compresses the at least one helical channel to produce the wave of compression.

28. A method of fabricating a pump, comprising the steps of:
(a) providing a master having a silicon wafer and a photoresist layer formed of a photoresist on the silicon wafer;
(b) exposing the photoresist layer to UV light through a patterned mask to cross-link the photoresist in selected regions in accordance with the mask to define channel regions;
(c) spin-coating the master with polydimethylsiloxane (PDMS) to form a PDMS layer that covers the defined channel regions thereon to form a rectangular, ribbon-shaped section encompassing channels; and
(d) plasma-bonding the PDMS layer having the channels to a PDMS film coated on a blank wafer to encapsulate the channels.

29. The method of claim 28, further comprising the step of wrapping the encapsulated channels around a cam shaft in a single helical turn to form the pump.

30. The method of claim 29, wherein the cam shaft has M fins spaced-apart formed on its exterior surface along its axis, such that when the cam shaft driven by the cam rotates, the M fins of the cam shaft compresses one or more of the encapsulated channels to produce a wave of compression that actuates a peristaltic flow of fluid therein.

31. The method of claim 28, further comprising the step of engaging with the encapsulated channels with an exterior threaded rod to form the pump, such that when the threaded rod rotates, the ridge of the threaded rod compresses one or more of the encapsulated channels to produce a wave of compression that actuates a peristaltic flow of fluid therein.

32. The method of claim 28, further comprising the step of baking and developing the defined channel regions to form a hardened, reusable master, prior to the spin-coating step (c).

33. The method of claim 28, wherein the spin-coating step comprises the steps of:
(a) peeling off the rectangular ribbon-shaped section of the PDMS layer;
(b) placing the peeled PDMS layer inversely on a glass slide;
(c) plasma-treating the peeled PDMS layer and the PDMS film coated on the blank wafer for a period of time;
(d) boding the peeled PDMS layer and the PDMS film coated on the blank wafer together such that the channels are encapsulated;
(e) removing the glass slide; and
(f) cutting the encapsulated channels from the blank wafer to form a rectangular ribbon-shaped piece of PDMS.

34. A system for manipulation of cells, comprising:
(a) at least a first pump and a second pump, each pump comprising:
at least one fluidic flow channel having an inlet and an outlet; and
means rotatably engaged with the at least one fluidic flow channel for periodically producing a wave of compression in the at least one fluidic flow channel, wherein the wave of compression actuates a peristaltic flow of fluid in the at least one fluidic flow channel so as to direct a desired amount of the fluid toward one of the inlet and the outlet; and
(b) a downstream device comprising:
a plurality of cell traps arranged in a square trap chamber defining a trap region thereof; and
a network of binary flow splitters,
wherein each side of the trap region is connected to the network of binary flow splitters that divides or gathers a flow uniformly across the entire side, wherein the network of binary flow splitters has a first input and a first output along the x direction and a second input and a second output along the y direction perpendicular to the x direction,
wherein the inlet and the outlet of the first pump are respectively in fluid communications with the first input and the first output of the network of binary flow splitters, and the inlet and the outlet of the second pump are respectively in fluid communications with the second input and the second output of the network of binary flow splitters, such that in operation, the first pump and the second pump drive orthogonal flow streams in the downstream device, thereby positioning cells to a desired location in the trap region.

35. The system of claim 34, wherein the means comprises an external threaded rod such that when the threaded rod rotates, the ridge of the threaded rod compresses the at least one fluidic flow channel to produce the wave of compression.

36. The system of claim 34, wherein the means a cam having a cam shaft, wherein the cam shaft has an axis, an exterior surface and M fins spaced-apart formed on the exterior surface along the axis such that when the cam shaft driven by the cam rotates, the M fins of the cam shaft compresses the at least one helical channel to produce the wave of compression.

37. A system for fluidic impedance tomography (FIT), comprising:
(a) an object chamber;
(b) a common channel spaced-apart surrounding the object chamber;
(c) N pumps spaced-apart coupling between the object chamber and the common channel, each pump comprising:
at least one fluidic flow channel having an inlet and an outlet; and
means rotatably engaged with the at least one fluidic flow channel for periodically producing a wave of compression in the at least one fluidic flow channel, wherein the wave of compression actuates a peristaltic flow of fluid in the at least one fluidic flow channel so as to direct a desired amount of the fluid toward one of the inlet and the outlet; and
(d) N pressure transducers, each pressure transducer connected between the object chamber and the surrounding common channel and associated with a respective pumps for determining the pressure difference between the periphery of the object chamber and the surrounding common channel therein,
wherein the measurement of the pressure distribution P, for each of a series of flow Q, distributions is used to reconstruct a fluidic impedance distribution within the object chamber.

38. The system of claim 37, wherein a pattern of flow through the object chamber is determined by the rate and direction of the flow Q, driven by each pump, where 1≦i≦N, with the constraint
$${\sum }_{i = 1}^{N} {Q}_{i} = 0$$
that the fluid is incompressible and the at least one fluidic flow channel of each pump is non-distensible.

39. The system of claim 37, wherein the flow patterns are determined by the gradient of the pressure distribution that satisfies Laplace's equation with the boundary conditions associated with the injection or removal from the fluid and the distribution of fluidic impedance associated with the objects contained in the central chamber.

40. The system of claim 37, wherein the surrounding common channel has a channel depth adapted such that a fluidic impedance of the surrounding common channel is negligible relative to that of the object chamber.

41. The system of claim 37, wherein the means comprises an external threaded rod such that when the threaded rod rotates, the ridge of the threaded rod compresses the at least one fluidic flow channel to produce the wave of compression.

42. The system of claim 37, wherein the means a cam having a cam shaft, wherein the cam shaft has an axis, an exterior surface and M fins spaced-apart formed on the exterior surface along the axis such that when the cam shaft driven by the cam rotates, the M fins of the cam shaft compresses the at least one helical channel to produce the wave of compression.
